(19) United States
(12) Patent Application Publication
(10) Pub. No.: US 2014/0186912 A1
(43) Pub. Date: Jul. 3, 2014

(54) METHODS FOR IMPROVED ETHANOL PRODUCTION

(71) Applicant: Board of Trustees of the Southern Illinois University on Behalf of Southern Illinois Universi, Edwardsville, IL (US)

(72) Inventor: Sabrina Trupia, Edwardsville, IL (US)

(21) Appl. No.: 14/146,506

(22) Filed: Jan. 2, 2014

Related U.S. Application Data
(60) Provisional application No. 61/748,374, filed on Jan. 2, 2013.

(51) Int. Cl. C12P 7/14

(52) U.S. Cl. CPC C12P 7/14
USPC 435/162; 435/294.1

(57) ABSTRACT

Some embodiments provide a process for the production of ethanol. The process includes performing liquefaction, saccharification, and fermentation steps to produce ethanol from an organic material. In one embodiment, the liquefaction step may include disposing a carbon-containing material within a first vessel, adding at least one enzyme to the first vessel, and incubating the first vessel at a first temperature for a predetermined time period. Next, the saccharification step can include adding at least one enzyme to the first vessel and then incubating the first vessel at a second temperature to form a liquefact in the first vessel. Then, in some embodiments, the fermentation step includes removing the liquefact from the first vessel and transferring the liquefact to a second vessel. Once in the second vessel, at least one additional enzyme, at least one nutrient, and a plurality of yeast cells can be added to the second vessel. Then, the second vessel is incubated to promote fermentation.

### CROSS-REFERENCE TO RELATED APPLICATIONS

This application claims priority to U.S. Application No. 61/748,374 filed Jan. 2, 2013, the entire disclosure of which is herein incorporated by reference.

### GOVERNMENTAL SUPPORT

This invention was made with government support under 761665/ARS awarded by the United States Department of Agriculture—Agricultural Research Service and 763945/11-041002 awarded by the Illinois Department of Commerce and Economic Opportunity. The government has certain rights in the invention.

### FIELD

The present invention generally relates to the production of ethanol from a biomass and is particularly related to an improved process for increased ethanol production from a biomass using conventional and/or novel yeast strains.

### BACKGROUND

Biofuels are an attractive alternative to current petroleum-based fuels. For example, many biofuels can be used as fuel sources for transportation fleets with little or no change to current technologies and these fuels exhibit significant potential to improve sustainability and reduce greenhouse gas emissions. However, in order to meet goals for renewable biofuels, it may be useful to expand beyond using solely conventional sugar and starch-based crops. In particular, it may be useful to begin using additional feedstocks, such as lignocellulosic biomass, in addition to or in lieu of conventional feedstocks.

### SUMMARY

Some embodiments of the invention provide a process for the production of ethanol. The process may include performing liquefaction, saccharification, and fermentation steps to produce ethanol from an organic material. In one embodiment, the liquefaction step may include disposing an untreated carbon-containing material within a first vessel, adding at least one enzyme to the first vessel, and incubating the first vessel at a first temperature for a predetermined time period. Next, the saccharification step can include adding at least one enzyme to the first vessel and then incubating the first vessel at a second temperature to form a liquefact in the first vessel. Then, in some embodiments, the fermentation step includes removing the liquefact from the first vessel and transferring the liquefact to a second vessel. Once in the second vessel, at least one additional enzyme, at least one nutrient, and a plurality of yeast cells can be added to the second vessel. Then, the second vessel can be incubated to promote fermentation.

Additional objectives, advantages and novel features will be set forth in the description which follows or will become apparent to those skilled in the art upon examination of the drawings and detailed description which follows.

### BRIEF DESCRIPTION OF THE DRAWINGS

FIG. 1 is a simplified flow chart detailing steps of one embodiment of an ethanol-production process;

FIG. 2 is a graph showing results of an experiment testing the total concentration of solids during a liquefaction step of one embodiment of an ethanol-production process;

FIG. 3 is a graph showing results of an experiment testing the total concentration of solids during a fermentation step of the ethanol-production process of FIG. 2;

FIG. 4 is a graph showing results of an experiment testing the concentration of sugars, excluding xylose, during the course of the ethanol-production process of FIG. 2;

FIG. 5 is a graph showing results of experiments testing the concentration of sugars and ethanol during the course of the ethanol-production process of FIG. 2;

FIG. 6 is a graph showing results of experiments testing the concentration of sugars and ethanol during the course of another embodiment of the ethanol-production process;

FIG. 7 is a graph showing results of an experiment measuring average mass loss (in grams) during the course of another embodiment of the ethanol production process, with each data point representing an average of eight replicate measurements;

FIG. 8 is a graph showing results of an experiment measuring average mass loss (in grams) after 46 hours and 107 hours of fermentation during the course of the ethanol production process of FIG. 7, with the error bars representing one standard deviation of eight independent experimental units;

FIG. 9 is a graph showing results of an experiment measuring ethanol concentration (in percent weight of ethanol per total volume) after 46 hours and 107 hours of fermentation during the course of the ethanol production process of FIG. 7, with the error bars representing one standard deviation of eight independent experimental units;

FIG. 10 is a graph showing results of an experiment measuring ethanol yield (in grams of ethanol produced per grams of dry ground corn) after 46 hours and 107 hours of fermentation during the course of the ethanol production process of FIG. 7, with the error bars representing one standard deviation of eight independent experimental units;

FIG. 11 is a graph showing results of an experiment measuring starch consumption on a dry-weight basis (in grams of starch per 100 g) after completion of the ethanol production process of FIG. 7; and

FIG. 12 is a graph showing results of an experiment measuring the total fiber content of the reactions, including acid detergent fiber (ANF) and neutral detergent fiber (NDF) (in grams) after completion of the ethanol production process of FIG. 7.

Corresponding reference characters indicate corresponding elements among the view of the drawings. The headings used in the figures should not be interpreted to limit the scope of the claims.

### DETAILED DESCRIPTION

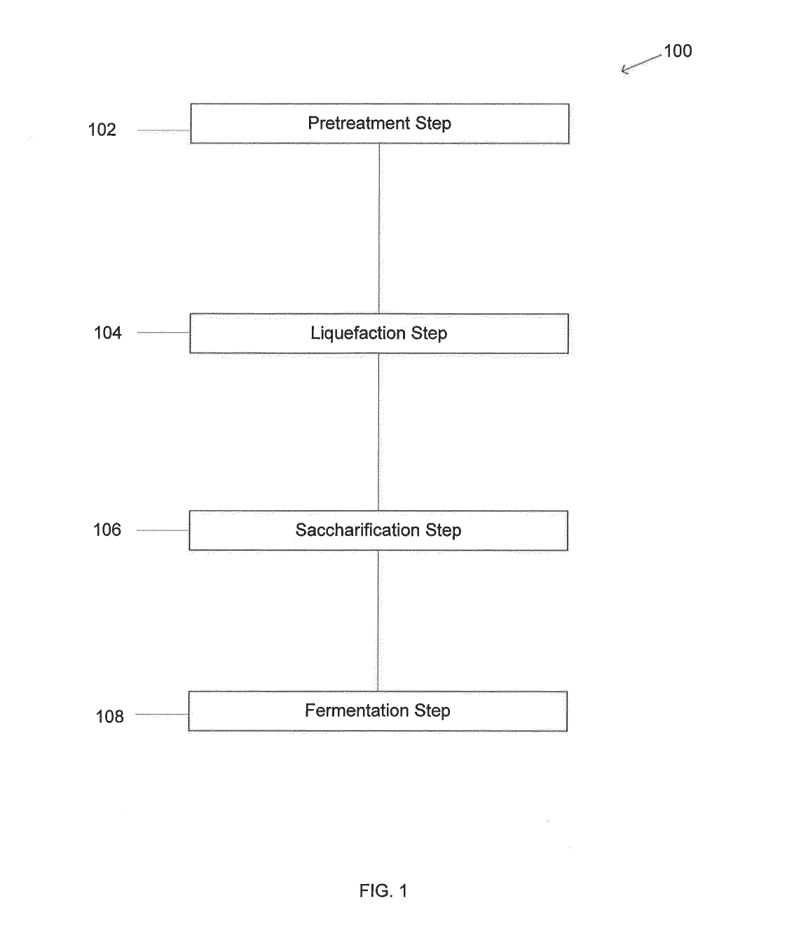

Referring to the drawings, an embodiment of an ethanol-production process is illustrated and generally indicated as 100 in FIG. 1. The ethanol-production process 100 can be employed to convert biomass-derived organic materials into materials that may be suitable for use as energy sources. For example, the ethanol-production process 100 can be used in converting five- and six-carbon compounds, such as xylose and glucose into a biofuel, such as ethanol. In one embodiment, the ethanol-production process 100 may be used in conjunction with biomass materials, such as corn, grass, wood, and other similar materials in the initial production of organic materials for the downstream production of biofuels. Moreover, in some embodiments, as discussed in further detail below, the ethanol-production process 100 can be used in conjunction with one or more fermentative organisms for the production of the biofuel from the biomass-derived organic material. As a result, some embodiments of the invention can be used in meeting at least some of the biofuel goals for the global economy in the coming years.

In the following discussion and examples, in some embodiments, corn-based products (e.g., non-milled corn fiber or corn fiber from wet and/or dry mill corn ethanol plants) can be used as sources of carbon-containing materials for use in biofuel production. However, in other embodiments, in addition to, or in lieu of the corn-based products, other organic materials can be processed for the release of carbon-containing materials for biofuel production. For example, any of the following materials can be used with or in place of corn-based products: woody biomass, forage grasses, herbaceous energy crops, non-woody plant biomass, agricultural wastes and/or agricultural residues, forestry residues and/or forestry wastes, paper-production sludge and/or waste paper sludge, waste-water-treatment sludge, municipal solid waste, and sugar-processing residues (i.e., any lignocellulosic material or cellulosic biomass can be used with the ethanol-production process 100).

In some embodiments, the ethanol-production process 100 can include a plurality of steps. For example, in one embodiment, the ethanol-production process 100 can include an optional pretreatment step 102, a liquefaction step 104, a saccharification step 106, and a fermentation step 108. As discussed in greater detail below and in the Examples section, one or more of the steps of the ethanol-production process 100 can occur in one or more pots, vessels, or other structures capable of providing the necessary structural support and environmental conditions needed for the ethanol-production process 100. In addition, in some embodiments, one or more of the above-mentioned steps can occur simultaneously (e.g., liquefaction and saccharification steps 104, 106) or can be repeated one or more times to produce the desired results. Moreover, as mentioned above, corn-based materials are used below to illustrate some embodiments of the ethanol-production process 100; however, any other of the above-mentioned materials, or the like, can be used in conjunction with the ethanol-production process 100 to produce ethanol.

In some embodiments, the optional pretreatment step 102 can be the first step of the ethanol-production process 100. In other embodiments, the pretreatment step 102 can occur simultaneously with or after one or more of the other steps of the ethanol-production process 100. In yet other embodiments, the ethanol-production process 100 can be implemented without a pretreatment step 102.

For example, in some embodiments, the optional pretreatment step 102 occurs before the other steps in the ethanol-production process 100. The optional pretreatment step 102 can be used to initially physically and/or mechanically disrupt the corn-based product to begin release of the carbon-containing materials. Accordingly, in one embodiment, the optional pretreatment step 102 may be used before the downstream lytic and fermentative steps. For example, the optional pretreatment step 102 can include mechanical stress applied to the corn-based product at a relatively high temperature. In one embodiment, the mechanical stress can be applied using one or more pressurized liquid streams (e.g., water streams). For example, the optional pretreatment step 102 may occur in a reactor (e.g., a Littleford Reactor) using pressurized water at a heated temperature (e.g., around 160° Celsius) for a predetermined period of time (e.g., about twenty-five minutes).

In some embodiments, the liquefaction step 104 and/or the saccharification step 106 can occur after the optional pretreatment step 102. In other embodiments, the liquefaction step 104 and/or the saccharification step 106 can occur without an optional pretreatment step 102. In this aspect, untreated carbon-containing material may be placed within a first vessel for the liquefaction and saccharification steps 104, 106. The ethanol-production process 100 includes liquefaction and saccharification steps 104, 106 to process the untreated carbon-containing material to release greater amounts of carbon-containing materials for downstream biofuel production. For example, the liquefaction and saccharification steps 104, 106 occur at substantially the same time in the same vessel. In particular, untreated carbon-containing material can be disposed in a first vessel (e.g., a bioreactor) with a volume of liquid (e.g., water) to form a substrate solution. The liquid can be used to ensure that the substrate solution sufficiently, substantially, or completely fills the bioreactor, thereby providing substantial or complete contact with a thermal jacket of the bioreactor. In addition, the liquid can dilute the substrate solution to a degree that the substrate solution can be readily agitated or stirred in a relatively efficient manner. For example, the vessel may include a mechanical actuating apparatus (e.g., an impeller) to stir the substrate solution during the liquefaction and saccharification steps 104, 106.

In one embodiment, in addition to being stirred during the liquefaction and saccharification steps 104, 106, one or more proteinaceous materials may be added to further enhance release of one or more carbon-containing materials. In one embodiment, a plurality of enzymes can be added to the substrate solution to improve the breakdown of the substrate within the substrate solution. By way of example only, the enzymes can include cellulase (i.e., enzymes that hydrolyze cellulose), β-glucosidase, xylanase, hemicellulase, any combination thereof, and/or any other enzymes that can further breakdown the material constituting the substrate. In other embodiments that include the use of materials other than corn as the substrate, the mixture and selection of enzymes will vary according to the constituent materials and carbohydrates contained therein.

After the addition of the plurality of enzymes, the bioreactor may be substantially or completely sealed and the mechanical actuating apparatus can be activated to begin stirring the substrate solution. For example, the mechanical actuating apparatus can stir the substrate solution at a rate of between 100 to 350 revolutions per minute (RPM). Moreover, the thermal jacket can be used to adjust the temperature within the bioreactor to a desired temperature (e.g., a temperature between 40° and 200° Celsius). The liquefaction and saccharification steps 104, 106 can occur for a predetermined period of time (e.g., around twenty-four hours). After completion of the liquefaction and saccharification steps 104, 106, the material contained within the bioreactor is known as the liquefact.

In other embodiments, the liquefaction and saccharification steps 104, 106 can occur as two distinct steps. In one embodiment, the ethanol-production process 100 can include the liquefaction step 104 as the initial step. For example, corn product (e.g., corn kernels) can be cleaned, milled and added with a liquid (e.g., water) to a vessel to form a slurry. In addition, during the liquefaction step 104, an enzyme, such as an alpha-amylase enzyme, may be added to the vessel as well. The liquefaction step 104 can then proceed to a heating and agitation step, where, for example, the slurry can be substantially or completely continuously mixed for a predetermined amount of time (e.g., around one to two hours) at a predetermined temperature (e.g., around 50° to 100° Celsius). After the predetermined time period, the vessel and slurry can be cooled for later steps (e.g., can be cooled to about 40° Celsius).

In some embodiments, the saccharification step 106 can occur in the same vessel, but as a substantially distinct step in the ethanol-production process 100. For example, after completion of the liquefaction step 104 discussed above, one or more of the previously mentioned enzymes can be added to the vessel, which can then be incubated with substantially or completely continuous mixing for a predetermined amount of time (e.g., around one to two hours) at a predetermined temperature (e.g., around 50° to 100° Celsius). After the predetermined time period, the vessel and slurry can be cooled for later steps (e.g., can be cooled to about 40° Celsius) to give rise to the liquefact.

In some embodiments, the fermentation step 108 occurs after the liquefaction and/or saccharification steps 104, 106. For example, as described in greater detail below, the fermentation step 108 includes the use of a conventional, industrial grade yeast strain (e.g., Ethanol Red provided by Fermentis) and/or a genetically-modified yeast strain that can ferment non-traditionally fermentable sugars, such as xylose, in addition to other sugars, such as glucose. In addition, in some embodiments, the yeast strain can be of the Saccharomyces cerevisiae lineage; however, in other embodiments, the yeast can be of a different species or genus and species (e.g., bacterial, protozoan, or other eukaryotic organisms).

In some embodiments, the fermentation step 108 can occur at a lower temperature than the liquefaction and saccharification steps 104, 106. For example, after substantial completion of the liquefaction and saccharification steps 104, 106, the temperature of the thermal jacket can be set to a temperature that is suitable for the yeast (e.g., around 30° Celsius). As a result, after a period of time, the temperature of the liquefact would be suitable to sustain growth of the yeast. In one embodiment, after reaching a sufficient temperature, a plurality of fermentation enzymes and a plurality of nutrients can be added to the liquefact to provide a suitable environment for fermentation. For example, some or all of the enzymes mentioned above can be added to the liquefact, in addition to or in lieu of other enzymes such as glucoamylases and other enzymes. Moreover, nutrients such as nicotinic acid, thiamine, and ammonium chloride can be added to the liquefact. In addition, in some embodiments, one or more compounds with antibiotic properties (e.g., bacteriostatic or bacteriocidal) can be added to substantially prevent or reduce any non-desirous growth within the bioreactor. In addition, between 1 and 2×10⁷ yeast cells can be added to the liquefact to initiate fermentation.

In some embodiments, the fermentation step 108 can occur for a predetermined amount of time under defined conditions. For example, the fermentation step 108 can occur at a constant temperature of about 30° Celsius and a constant pH of approximately 4.0 for between about 24 and 120 hours. During the fermentation step 108, one or more samples can be extracted from the bioreactor to assess the progress of the fermentation step 108 and determine the relative concentrations of constituents within the fermenting liquefact.

As discussed in the following examples, one or more of these above-discussed steps can be varied or duplicated as needed to optimize and/or enhance the ethanol-production process 100.

### EXAMPLES

The following discussion is intended only as examples of some embodiments of the invention. The details set forth below are not intended to limit the scope of the disclosure, but are rather to provide exemplary uses of some embodiments of the invention.

### Example 1

### 30 Liter Industrial-Scale Fiber Fermentation

An initial experimental series was conducted to assess the conversion of organic material to ethanol by an industrial-grade yeast on a generally industrial scale. For instance, the following experiments were executed to address at least the following parameters (i) capability of a pretreatment to adequately prepare a corn-based product (referred to herein as corn bran, corn fiber, or substrate) for liquefaction and fermentation; and (ii) capability of an experimental set of cellulosic enzymes to substantially or completely liquefy hemicellulosic corn fiber and adequately liberate sugars for fermentation.

Experimental Design

In general, pretreated corn fiber was liquefied and fermented using experimental enzymes and an industrial-grade yeast in a 30 Liter (L) bioreactor in a pilot plant over a period of approximately 3 days. Table 1 describes the timeline of this experiment.

TABLE 1
Plan and conditions for Experiment 1.

| | | | |
|---|---|---|---|
| Day 1 | Day 2 | Day 3 | Day 4 |

| | |
|---|---|
| 50° C. | 32° C. |

| | | |
|---|---|---|
| 24-hour | 48-hour | Termination |
| Liquefaction | fermentation | of 48-hour |
| | | Fermentation |

Initially, the corn-based product was pretreated. In particular, the corn-based product was disposed in a Littlerford reactor and exposed to 160° C. water for 25 minutes to provide an initial mechanical stress to disrupt some of the superstructures of the corn product. The pretreatment process produced 16.18% solids weight per weight (w/w) hot-water treated corn fiber (PTCF) material.

After the pretreatment step, the PTCF material underwent liquefaction and saccharification. Specifically, the PTCF material was added to a bioreactor that included a Rushton impeller, a removable headplate, and pH and dissolved oxygen probes. Initially, the pH and dissolved oxygen were calibrated in advance of filling the bioreactor and were used throughout the process. Then, the headplate of the bioreactor was removed to allow 13.060 kg of 16.18% solids (w/w) PTCF material to be disposed within the bioreactor, along with 6.420 kg water, which was added via a peristaltic pump through a headplate port once the headplate was reattached. The mixture of the water and the PTCF material formed a substrate solution. Furthermore, as previously mentioned, water was added for two purposes: (i) to ensure that the substrate/PTCF material would sufficiently fill the bioreactor so that it was in full-contact with a thermal jacket lining the bioreactor; and (ii) so that the substrate solution would be thin enough for efficient agitation.

Next, a plurality or cocktail of enzymes was added to the bioreactor through the headplate port via a syringe to advance the liquefaction and saccharification steps As shown in Table 2, these enzymes included Cellulase Complex (provided by Novozymes, catalog number NS22086) at a dose of 5% (w/w),β-glucosidase (provided by Novozymes, catalog number NS22118) at a dose of 0.6% (w/w), Xylanase (provided by Novozymes, catalog number NS22083) at a dose of 0.25% (w/w), and Hemicellulase (provided by Novozymes, catalog number NS22002) at a dose of 2% (w/w). All enzyme dosages in this experiment were the maximum dosages recommended by their manufacturer, Novozymes.

TABLE 2
Enzyme cocktail added at beginning of 24-hour liquefaction and
saccharification reactions.

| | Added | Solution | |
|---|---|---|---|
| Enzyme | (mL) | Concentration | dosage |
| Cellulase | 106 | 1 g/mL | 5% w/w |
| Complex | | | |
| β- | 13 | 1 g/mL | 0.6% w/w |
| Glucosidase | | | |
| Xylanase | 5.3 | 1 g/mL | 0.25% w/w |
| Hemicellulase | 42 | 1 g/mL | 2% w/w |

After sealing the bioreactor with the substrate solution therewithin, the Rushton impeller began stirring the substrate at variable set-points throughout the course of the experiment. For example, the set-points ranged from 100 RPM to 350 RPM. Under these circumstances, the liquefaction and saccharification steps lasted for a period of about twenty-four hours beginning with the addition of the cocktail of enzymes. During this experiment, a heating element associated with the thermal jacket was found to be inoperable, thereby requiring the use of an external water bath to circulate water through the thermal jacket to increase and then maintain the temperature of the substrate solution at around 50° C. Shortly after starting the liquefaction and saccharification steps, the water bath was attached to the thermal jacket and internal temperature reached the target temperature of 50° C. within an hour.

Twenty-two hours after beginning the liquefaction and saccharification steps, the water bath, and correspondingly, the thermal jacket set point was lowered to 30° C. As a result, the temperature of the substrate solution (now, the liquefact) was lowered to the fermentation reaction-target temperature by the end of the twenty-four hour liquefaction and saccharification steps. Accordingly, at this time, the 48-hour fermentation reaction was initiated.

Initially, the fermentation reaction involved the liquefact being dosed with a plurality of fermentation enzymes and a plurality of nutrients, as shown in Tables 3A and 3B. As shown in Table 3A, the fermentation enzymes included Enzyme Complex (provided by Novozymes, catalog number NS22119) at a dose of 5.0% (w/w), Glucoamylase (provided by Novozymes, catalog number 22035) at a dose of 0.4% (w/w), Spirizyme Excel (provided by Novozymes) at a dose of 0.066% (w/w), Hemicellulase (provided by Novozymes, catalog number NS22002) at a dosage of 2% (w/w). As shown in Table 3B, the nutrients included Nicotinic acid (provided by Sigma) at a dose of 0.03 grams per liter (g/L), Thiamine HCl (provided by Sigma) at a dose of 0.03 g/L, Ammonium Chloride (provided by Sigma) at a dose of 1.0 g/L and the antibiotic Lactrol (provided by Phibro) at a dose of 0.50 parts per million (ppm). After adding the fermentation enzymes and nutrients, 41 mL of an Ethanol Red Yeast (provided by Fermentis) ADY solution. In this case, the yeast were added a concentration of 2.0×10⁷ cells per mL of liquefact. After the addition of the yeast, the fermentation reaction was allowed to proceed for approximately 48 hours.

TABLE 3A
List of enzymes used in fermentation reaction along with how
much was added to the reaction, their concentrations, and the
dosages used.

| | Added | Solution | |
|---|---|---|---|
| Enzyme | (mL) | Concentration | Dosage |
| Enzyme | 155 | 1 g/mL | 5% w/w |
| Complex | | | |
| Glucoamylase | 13 | 1 g/mL | 0.4% w/w |
| Spirizyme | 2 | 1 g/mL | 0.066% w/w |
| Hemicellulase | 42 | 1 g/mL | 2% w/w |

TABLE 3B
List of nutrients and antibiotic used in fermentation reaction along with
how much was added to the reaction, their concentrations, and the
dosages used.

| | Added | Solution | | |
|---|---|---|---|---|
| Reagent | (mL) | Concentration | dosage | |
| Nicotinic | 96.7 | 3.75 g/L | 0.03 | g/L |
| Acid | | | | |
| Thiamine | 4.8 | 75 g/L | 0.03 | g/L |
| HCl | | | | |
| Ammonium | 24.2 | 500 g/L | 1.0 | g/L |
| Chloride | | | | |
| Lactrol | 3.9 | 2.5 g/L | 0.50 | ppm |
| Ethanol | 41 | 0.22 g/mL | 20E\hat{ }6 | cells/mL |
| Red Yeast | | | | |

During the course of the fermentation reaction, the pH was maintained at approximately 4.0 using 2N Ammonium Hydroxide (provided by Ricca). As discussed in greater detail below in the Results section, time point samples were taken throughout the fermentation reaction and analyzed for the presence of ethanol as well the concentrations of substrates (glucose, DP2, DP3, DP4+, and xylose, where “DPx” represent glucose oligomers with “x” subunits) and products (ethanol, glycerol, lactic acid, and acetic acid) by high-performance liquid chromatography (HPLC). Samples were prepared for HPLC by centrifugation to remove large solids, followed by filtration through 0.45 micrometer (μm) syringe filters, and acidification to pH of approximately 2 by addition of sulfuric acid to a final concentration of 0.01 N. Samples were also subjected to total and dissolved solids analyses. Fermentation progress was monitored by recording the weight of the bioreactor throughout the experiment and attributing mass loss to production and escape of carbon dioxide.

Results

Initially, when quantifying this experiment, the mass of the bioreactor was considered. As shown in Table 4, the initial and final bioreactor weights at the start of fermentation (time=0) and at the end of fermentation (time=48 hours) were 108.200 kilograms (kg) and 107.220 kg, respectively. Accordingly, the theoretical yield of ethanol was about 200 grams.

TABLE 4
Mass-based ethanol yield calculations

| Initial | | | Yield of | Yield of |
|---|---|---|---|---|
| mass | final mass | MEtOH | EtOH, mass | EtOH, vol. |
| (g) | (g) | (g) | (g/g dry) | (gal/bu) |
| 108200.0 | 107220.0 | 200.0820235 | 0.112963651 | 0.228875052 |

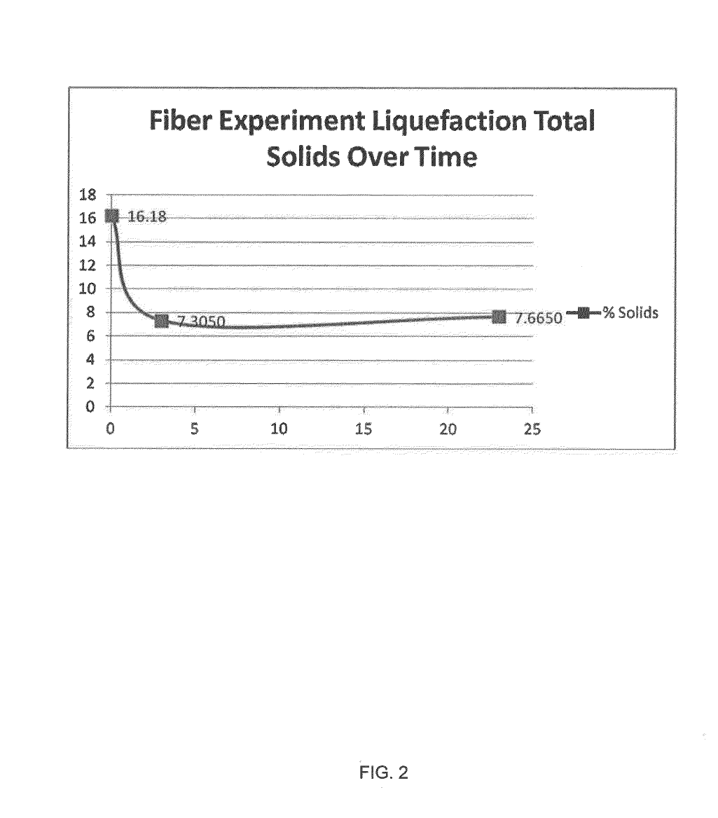

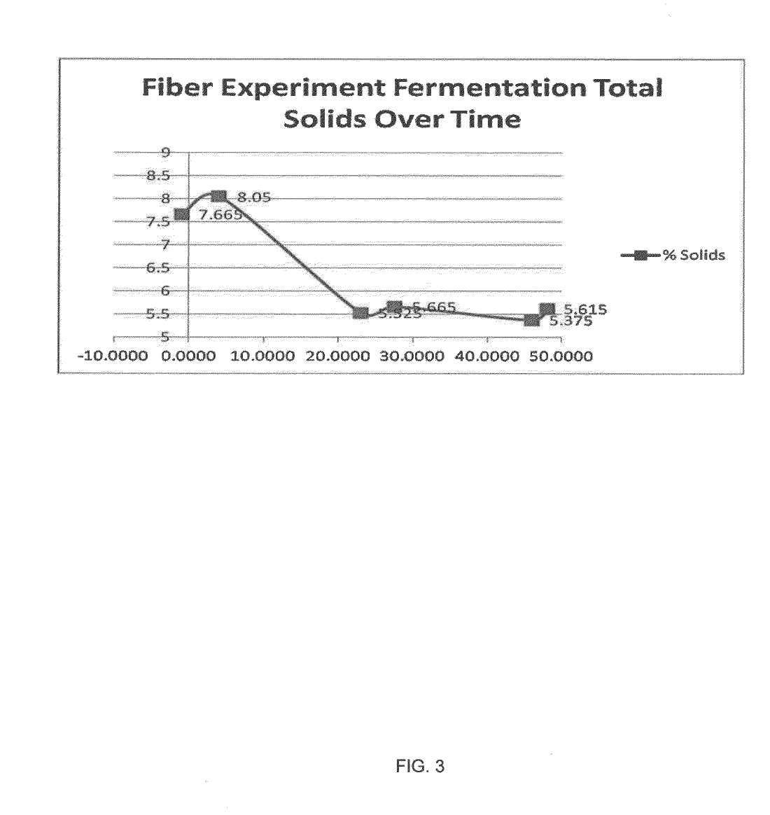

Referring now to FIG. 2, the total concentration of solids within the PTCF solution dropped, which is in accordance with the nature of the process. Specifically, the initial total solids of PTCF was determined to be 16.18%, which then fell to 7.66% after 23 hours of liquefaction, thereby supporting the idea that liquefaction step results in conversion of solid PTCF to liquid materials to be used for fermentation. Similarly, as shown in FIG. 3, the 23-hour liquefaction solids data also functions as the time point immediately prior the fermentation reaction because the 23-hour data point is one hour prior to the initiation of the fermentation reaction. After 48 hours of fermentation, the concentration of total solids within the bioreactor fell from 7.66% to 5.61%, which further supports the idea that the PTCF material was broken down from a solid phase to a liquid phase during the liquefaction and saccharification steps and further broken down during the fermentation steps, likely as a result of the addition of the plurality of enzymes.

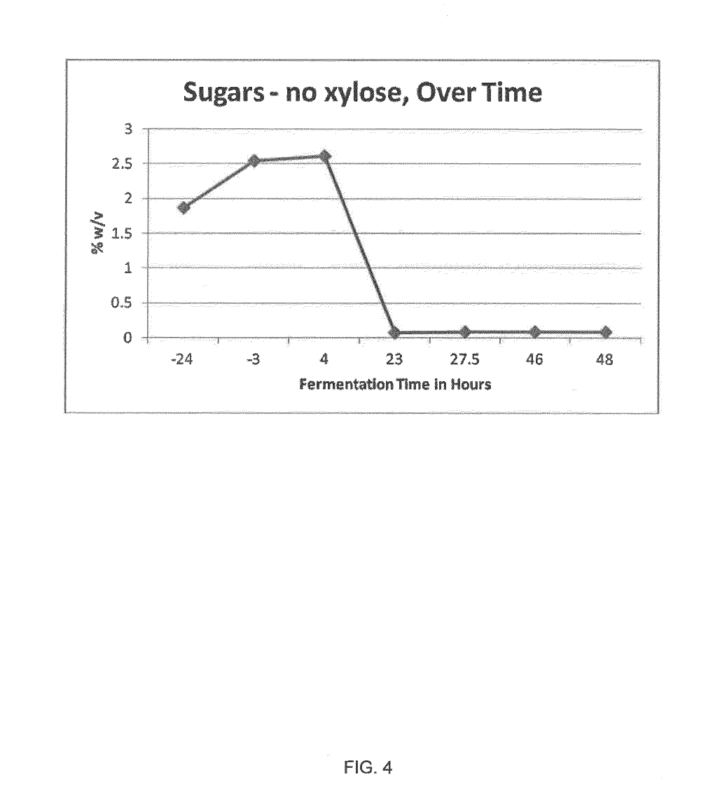

Next, as illustrated by the graph in FIG. 4, HPLC data shows that the concentration of sugars in the solution contained within the bioreactor also fell during the course of the process. Specifically, the HPLC data shows that the weight per volume (w/v) concentration of sugars (not including xylose) was approximately 2-2.61% during the initiation of the fermentation step (i.e., time=−24 hours to time=4 hours relative to the initiation of the fermentation step), which dropped to 0.078% w/v at 23 hours after the initiation of the fermentation step.

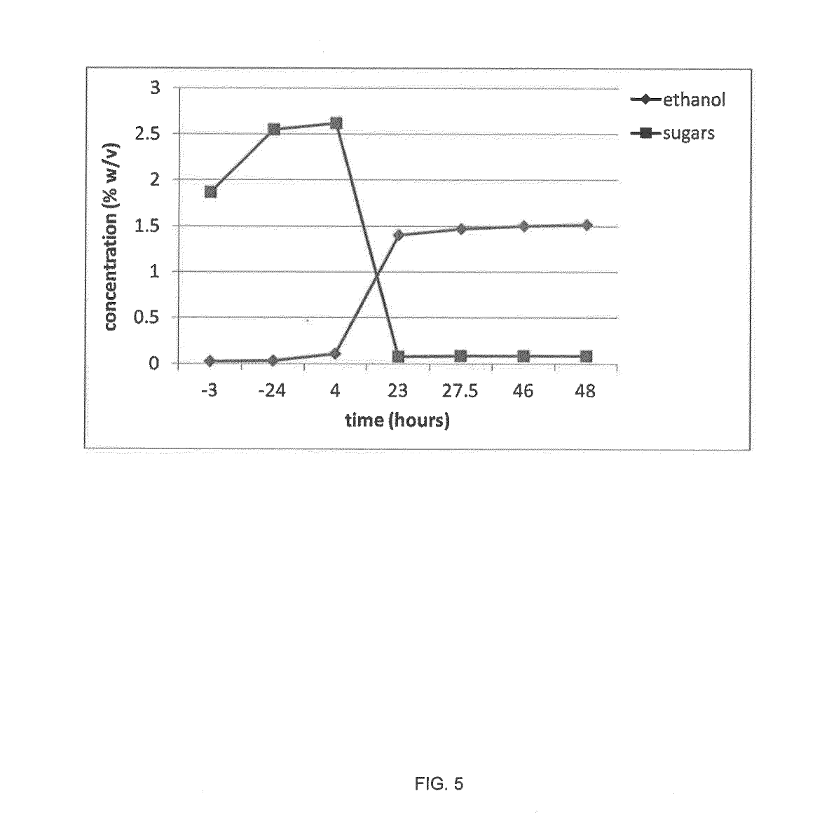

Referring now to FIG. 5, when contrasting sugar consumption and ethanol production during the course of the experiment, it is revealed that the ethanol-production process operated in accordance with expectations. Specifically, sugars were released from the PTCF material, thereby leading to an initial increase in a concentration of sugar within the solution prior to the initiation of the fermentation step. After the fermentation step begins, the concentration of sugars begins dropping and the concentration of ethanol rises.

Accordingly, this experiment aimed to demonstrate successful 24-hour liquefaction and saccharification steps and 48-hour fermentation of corn fiber in an industrial setting along with production of ethanol from sugars contained therewithin. Each of these goals were met and resulted in the production of 1.5% (w/v) ethanol using industrial-grade yeast. Using the above-described set-up, near-theoretical amounts of sugar were produced.

### Example 2

### 150 Liter Industrial-Scale Fiber Fermentation

Another experimental series was conducted to assess the conversion of xylose to ethanol using a genetically-modified yeast on a generally industrial scale. For instance, the following experiments were executed to address at least the following parameters (i) capability of a pretreatment to adequately prepare a corn-based product (referred to herein as corn bran, corn fiber, or substrate) for liquefaction and fermentation on a larger scale relative to Experiment 1; (ii) capability of an industrial-grade yeast to metabolize five-carbon containing sugars, such as xylose into ethanol on an industrial scale; and (iii) capability of an experimental set of cellulosic enzymes to substantially or completely liquefy hemicellulosic corn fiber and adequately liberate sugars for fermentation on a larger scale relative to Experiment 1.

Experimental Design

In general, pretreated corn fiber was liquefied and fermented using experimental enzymes and a genetically-modified yeast strain in a 150 L bioreactor in a pilot plant over a period of approximately two weeks. Table 5 describes the timeline of this experiment.

TABLE 5
Plan and conditions for Experiment 2.

| | | | | | | | |
|---|---|---|---|---|---|---|---|
| Day 1 | Day 2 | Day 3 | Day 4 | Day 5 | Day 6 | Days 7-11 | Day 12 |

30° C.

| | | | | | |
|---|---|---|---|---|---|
| 24-hour | 72-hour propagation. | Flasks | 24-hour | Sample at | Sampled at |
| yeast | | combined, | propagation in | every | 24-hour |
| propagation | | used to | 30 L, pumped | fermentation | mark, beer |
| in tubes. | | inoculate | to 150 L to | 24-hour | heat- |
| | | 30 L, Bran | inoculate | mark. | sterilized, |
| | | sterilized | liquefact. | | yeast killed. |
| | | and | | | |
| | | liquefied | | | |
| | | in 150 L. | | | |

Initially, during days 1-4 of the experiment, 10 vials of YRH400 glycerol stocks (30%, v/v) were retrieved from storage in a −80° C. freezer and thawed and then used to inoculate sterile SC5X broth and later, sterile YPD broth. YRH400 is a strain of Saccharomyces cerevisiae that has been genetically-modified to ferment xylose and other sugars to produce ethanol. All vessels containing SC5X broth used in this experiment were sterilized at 122° C. in an autoclave for 60 minutes.

These vials were incubated at 30° C. in an incubator/shaker, with agitation set at 150 RPM. After 24 hours, these vials were transferred to 10 sterile flasks (one tube per flask), with each flask containing 290 mL of sterile SC5X broth. These flasks were placed in an incubator/shaker for 72 hours (days 2-4 of the experiment), where they were incubated/shaken at 30° C. with agitation set at 150 RPM.

Starting on day 5, the corn-based product was pretreated or pre-treated corn-based product was obtained from a source. If pretreated in-house, the corn-based product was disposed in a Littlerford reactor and exposed to 160° C. water for 25 minutes to provide an initial mechanical stress to disrupt some of the superstructures of the corn product. The pretreatment process produced 15% solids w/w PTCF material.

After the pretreatment step, PTCF material underwent the liquefaction and saccharification steps. Specifically, the PTCF material was added to a bioreactor that included a Rushton impeller, a removable headplate, and pH and dissolved oxygen probes. Initially, the pH and dissolved oxygen were calibrated in advance of filling the bioreactor and were used throughout the process. Then, the headplate of the bioreactor was removed to allow 38.18 kg of 15% solids (w/w) PTCF material to be disposed within the bioreactor, along with 46.6 kg water.

After adding the water and PTCF, a plurality of enzymes was combined in a sterile hood in a sterile carboy, which included a sterile tubing apparatus and a sterile air filter. In addition, the bioreactor included at least two addition ports, which were kept sterile throughout the process by running steam across a sealed inlet valve, an open sample valve and through an open condensate line. Just prior to the time of addition of the enzymes, the steam was turned off and the condensate line closed so that the addition port would cool enough to not damage enzymatic function. In addition, steam was also circulated through the thermal jacket so that the substrate contained within was heated to 122° C. for at least one hour and then cooled back down to 40° C.

As shown in Table 6, the mixture of enzymes included multiple enzymes. Specifically, the mixture included Cellulase Complex (provided by Novozymes, catalog number NS22086) at a dose of 5% (w/w),β-glucosidase (provided by Novozymes, catalog number NS22118) at a dose of 0.6% (w/w), Xylanase (provided by Novozymes, catalog number NS22083) at a dose of 0.25% (w/w), and Hemicellulase (provided by Novozymes, catalog number NS22002) at a dose of 2% (w/w), and the antibiotic Lactrol (provided Phibro) at a dose of 0.50 ppm. All enzyme dosages in this experiment were the maximum dosages recommended by their manufacturer, Novozymes.

TABLE 6
Enzyme cocktail added at beginning of 24-hour liquefaction and
saccharification reactions.

| Reagent | Added | Dosage | |
|---|---|---|---|
| Cellulase | 286.41 g | 5% | w/w |
| Complex | | | |
| β- | 34.111 g | 0.6% | w/w |
| Glucosidase | | | |
| Xylanase | 14.446 g | 0.25% | w/w |
| Hemicellulase | 114.701 g | 2% | w/w |
| Lactrol | 7.6 mL | 0.50 | ppm |
| (2.5 g/L) | | | |

After sealing the bioreactor with the substrate solution therewithin, the Rushton impeller began stirring the substrate at a set point of about 200 RPM throughout the course of the experiment. Under these circumstances, the liquefaction and saccharification steps lasted for a period of about twenty-four hours beginning with the addition of the cocktail of enzymes. After all additions to the bioreactor were made, the addition port was reset so that the valves were sealed with steam running across the port from the steam supply line through the condensate line to ensure sterility. During the liquefaction and saccharification steps, the pH was maintained at around 5.0 through the use of 20% w/w ammonium hydroxide (provided by Ricca).

During the liquefaction and saccharification steps, the flasks containing the YRH400 were combined in a carboy equipped with tubing and sterile air filter in a sterile hood (e.g., a laminar-flow hood). The carboy and tubing assembly were sterilized at 122° C. in an autoclave for approximately 15 minutes. Next, a Applikon 30 L bioreactor was prepared for propagation by pumping 17 L of water into the bioreactor through a headplate port and 850 g of YPD broth powder (provided by Sigma) was also added through this port. A pressure-hold test was performed for 15 minutes. After passing the pressure-hold test, the reconstituted broth was sterilized for 45 minutes at 122° C., with agitation set at 350 RPM using a Rushton impeller, and then cooled to 30° C. before inoculation.

Next, the sterile carboy containing the YRH400 yeast in 3 L of SC5X broth was attached to an addition port on the headplate of the 30 L bioreactor. The addition port and tubing opening were thoroughly sprayed with 70% volume per volume (v/v) ethanol before attaching the tubing to the barb to prevent microbe contamination. A peristaltic pump was used to pump the inoculum (i.e., the YRH400 within the sterile carboy) into the 30 L bioreactor through the open port. The 30 L bioreactor was maintained at 30° C. with agitation at about 350 RPM for about 24 hours, at which time it was ready to inoculate the liquefact contained in the 150 L bioreactor.

After completion of the liquefaction and saccharification steps, the liquefact was inoculated with the YRH400 strain. The inoculation was done by attaching a sterilized (i.e., autoclaved at 122° C. for 30 minutes) transfer hose from a drain valve of the 30 L bioreactor to an inoculum port of the 150 L bioreactor. In order to ensure sterility, the hose was attached about six hours in advance of the transfer and steam (i.e., water vapor at 122° C.) ran through the hose during this time, which was supplied from the 30 L bioreactor and to the 150 L inoculum port condensate line. One hour prior to the transfer, the steam flow was shut off so that the line would cool enough to prevent destruction of yeast cells. The entire contents of the 30 L vessel were then transferred to the liquefact contained in the 150 L bioreactor, marking the initiation of the fermentation reaction (i.e., time=0 hours). In order to perform the transfer, air was used to pressurize the 30 L vessel. Correspondingly, pressure in the 150 L bioreactor had to be lower than the pressure contained within the 30 L vessel during this process to prevent blowback into the 30 L vessel. Accordingly, the pressure within the 30 L vessel was maintained at approximately 8 pounds per square inch (“psi”) and the pressure within the 150 L bioreactor was maintained at approximately 2 psi during the transfer. After the transfer was completed, all valves were closed in the reverse order of the described transfer and, to ensure sterility, the steam flow across the ports was reopened in the 150 L bioreactor.

An additional set of enzymes and nutrients were added to the liquefact. As shown in Table 7A, the set of enzymes added to the liquefact included Enzyme Complex (provided by Novozymes, catalog number NS22119) at a dose of 5.0% (w/w) and Glucoamylase (provided by Novozymes, catalog number 22035) at a dose of 0.4% (w/w), As shown in Table 7B, the nutrients included Nicotinic acid (provided by Sigma) at a dose of 0.03 g/L, Thiamine HCl (provided by Sigma) at a dose of 0.03 g/L, Ammonium Chloride (provided by Sigma) at a dose of 1.0 g/L and the antibiotic Lactrol (provided by Phibro) at a dose of 0.50 ppm. As described above, the YRH400 yeast was then added to the liquefact from the 30 L fermentor. The concentration of cell mass in the inoculum was estimated to be 1.3×10⁷ cells/mL. The solution was allowed to ferment for around 114 hours.

TABLE 7A
List of enzymes used in fermentation reaction along with
how much was added to the reaction, their concentrations,
and the dosages used.

| | Added | |
|---|---|---|
| Enzyme | (g) | Dosage |
| Enzyme | 85.9 | 5% w/w |
| Complex | | |
| Glucoamylase | 22.9 | 0.4% w/w |

TABLE 7B
List of nutrients used in fermentation reaction along with
how much was added to the reaction, their concentrations,
and the dosages used.

| | Added | Solution | |
|---|---|---|---|
| Reagent | (mL) | Concentration | dosage |
| Nicotinic | 263 | 3.75 g/L | 0.03 g/L |
| Acid | | | |
| Thiamine | 14 | 75 g/L | 0.03 g/L |
| HCl | | | |
| Ammonium | 236 | 500 g/L | 1.0 g/L |
| Chloride | | | |

During the experiment, the pH was maintained at approximately 5.2 through the addition of 2N ammonium hydroxide (Ricca). Time point samples were taken throughout the experiment and analyzed for the presence of ethanol as well the concentrations of substrates (glucose, DP2, DP3, DP4+, and xylose, where “DPx” represent glucose oligomers with “x” subunits) and products (ethanol, glycerol, lactic acid, and acetic acid) by HPLC. Samples were prepared for HPLC by centrifugation to remove large solids, followed by filtration through 0.45 μm syringe filters, and acidification to a pH of approximately 2 by addition of sulfuric acid to a final concentration of 0.01 N. Samples were also tested for total and dissolved solids analyses. Fermentation progress was monitored by recording the weight of the vessel throughout the experiment and attributing mass loss to the production and escape of carbon dioxide.

Results

Initially, like Experiment 1, the total solids of the PTCF material was determined to assess the progress of organic-material lysis and liquefaction. Specifically, the concentration of total solids of the PTCF material was initially 13.3275% and then fell to 5.49% after 24 hours of the liquefaction step. In addition, after 114 hours of fermentation, total solids fell from 5.49% to 4.16% (data not shown). Accordingly, like Experiment 1, the above-described process leads to the reduction in concentration of solid PTCF material and the production of liquefied organic material.

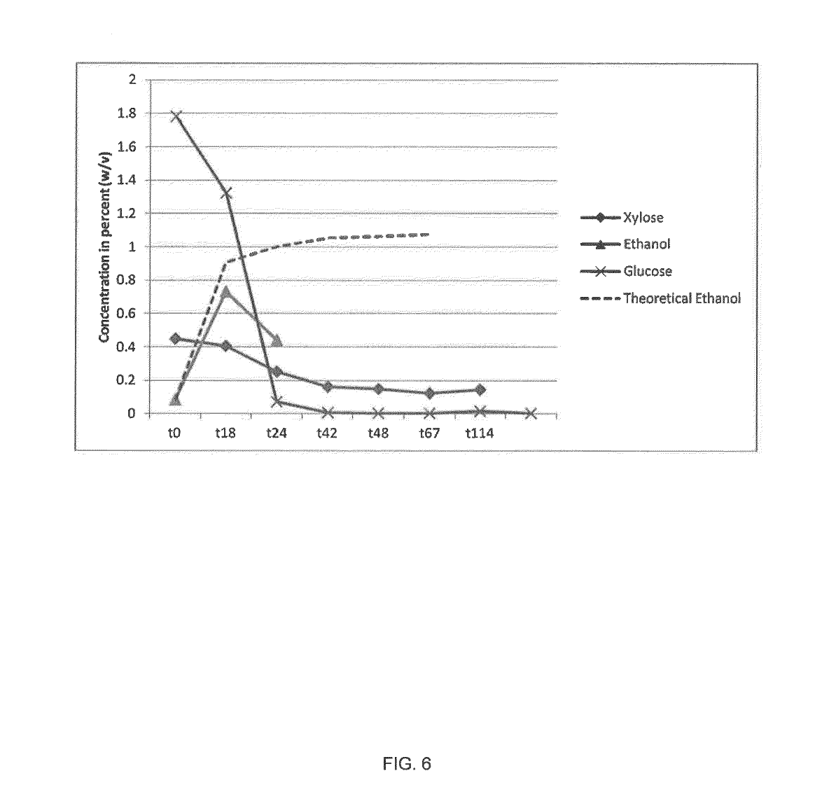

Next, referring to FIG. 6, HPLC data illustrates that the above-described process results in the fermentation of both glucose and xylose. Specifically, the HPLC data show a 0.4486% (w/v) concentration of xylose at the initiation of the fermentation reaction (time=0). After 67 hours of fermentation, however, the concentration of xylose fell to 0.1239% (w/v). In addition, ethanol was produced at a level of 0.73% (w/v) after 18 hours of fermentation. Ethanol levels later dropped to 0.44% (w/v) after 24 hours of fermentation and then fall below detectable levels for the remainder of the experiment. The loss of ethanol detection is likely due to ethanol evaporation through the 150 L-bioreactor “off-gas” line.

Accordingly, this experiment demonstrated a successful 24-hour liquefaction and saccharification, with an extended fermentation reaction using a genetically-modified yeast strain. These experiments were a success because of the production of 0.44% (w/v) ethanol using genetically-modified yeast with the capability to ferment xylose.

Experiment 3

Another experimental series was conducted to assess the conversion of xylose to ethanol by a genetically-modified yeast on a generally industrial scale. For instance, the following experiments were executed to address at least the following parameters (i) whether the use of cellulosic enzymes will result in an increased production of ethanol using a conventional industrial-grade yeast strain; (ii) whether only certain cellulosic enzymes are necessary to provide sufficient sugar content for ethanol production; and (iii) the optimal time to complete fermentation using cellulosic enzymes.

Experimental Design

In general, the following experiments involved one control and two treatments or experimental groups. All controls and treatments were tested in eight independent replicates, for a total of 24 units. Table 8 illustrates the treatments that were tested and their respective compositions.

TABLE 8
Summary of experimental design for Experiment 3.

| | Labomat 1 | Labomat 2 | |
|---|---|---|---|
| | (85° C., 90 min) | (50° C., 24 hours) | |
| Type | (Liquefaction) | (Saccharification) | Fermentation |
| Control | Alpha Amylase | Lactrol (hold) | Glucoamylase |
| | | | (Spirizyme |
| | | | Excel), Ethanol |
| | | | Red, |
| Treatment 1 | | Cellulase complex | Glucoamylase |
| | | NS22806, Beta- | (NS22035), |
| | | glucanase, | Ethanol Red |
| | | NS22118, and | |
| | | Lactrol | |
| Treatment 2 | | Cellulase complex | |
| | | NS22086, Beta- | |
| | | glucanase, | |
| | | NS22118, | |
| | | Xylanase | |
| | | NS22083, Enzyme | |
| | | Complex | |
| | | NS22119, | |
| | | Hemicellulase | |
| | | NS22002, and | |
| | | Lactrol | |

In this experiment, the initial step of the reaction was the liquefaction step. However, prior to liquefaction, the corn-based product was cleaned and milled. Specifically, large debris was removed from the standard corn by hand, and small debris (<4 millimeters (mm)) was removed by passing the corn over a No. 5 sieve (provided by Fisher Scientific Company) before grinding using conventional operating procedures. Specifically, the grinding method used a Wiley Mill grinder (provided by Thomas Scientific, NJ) with a 2 mm screen. Next, the moisture content of the ground corn was gravimetrically determined using a forced-air oven and measuring any mass loss that occurred as a result of the drying.

Using predetermined ratios, the mass of corn, the volume of water to be added, and weights and concentrations of enzyme needed to prepare a 200 g of corn slurry at a total dry solids concentration of 30% (w/w) for each replicate was determined. For each treatment, eight independent replicate slurries were prepared by weighing the required amount of deionized water into labeled Labomat beakers (provided by Labomat or Uys Fick Engineering C.C.) followed by the addition of the required mass of ground corn. The liquefaction step was carried out using an alpha-amylase enzyme (Liquozyme SC DS, provided by Novozymes). Specifically, the alpha-amylase enzyme was diluted to provide for a more-precise delivery of enzyme to each beaker. For example, a 0.15 g/mL working solution of the alpha-amylase stock was used and added at a dose of 0.025% (w/w) based on the wet weight of the corn flour.

Next, the slurries were hand swirled after all components were added to the Labomat beakers. The Labomat beakers were then sealed and attached to a vertically-mounted wheel in a conventional Labomat system (Model BFL12 805, Mathis, Switzerland), which rotated at approximately 50 RPM during the incubation. The system was programmed to reverse direction of the wheel approximately every 50 seconds to improve the mixing efficiency. The liquefaction step proceeded by incubating the samples at 85° C. for about 90 minutes, after which the samples were cooled to 40° C. in the Labomat apparatus.

Next, the samples underwent the saccharification steps in the same Labomat beakers. As shown in Table 9 below, the different treatments and the control received different enzymes. Specifically, treatment 1 received received cellulase complex (provided by Novozymes, catalog number NS22086) at a dose of 5% (w/w) and β-glucanase (provided by Novozymes, catalog number NS22118) at a dose of 0.6% (w/w). Treatment 2 received cellulase complex and β-glucanase at the same dosages as treatment 1, in addition to xylanase (provided by Novozymes, catalog number NS22083) at a dosage of 0.25% (w/w), enzyme complex (provided by Novozymes, catalog number NS22119) at a dosage of 5% (w/w), and hemicellulase (provided by Novozymes, catalog number NS22002) at a dosage of 2% (w/w). As recited above “enzyme complex” includes a range of carbohydrases, including arabinases, beta-gluconase, cellulose, hemicellulase, pectinase, and xylanase.

TABLE 9
Summary of enzyme additions and amendments for each treatment.

| | | | | | | | | Spirizyme | | |
|---|---|---|---|---|---|---|---|---|---|---|
| | Cellulase | B- | | Enzyme | Hemicellulase | 2.5 mg/ml | 0.2 g/ml | Excel | Gluco | |
| | Complex | Glucosidase | Xylanase | Complex | dose | Antimicrobial | Urea | dose | dose | Yeast |
| Treatment # | (uL) | (uL) | (uL) | dose (uL) | (uL) | dose (uL) | (uL) | (uL) | (uL) | (uL) |
| Control | — | — | — | — | — | 40 | 1069 | 526 | — | 322 |
| Treatment | 5023 | 597 | — | — | — | 40 | 1069 | — | 414 | 322 |
| #1 | | | | | | | | | | |
| Treatment | 5023 | 597 | 275 | 5023 | 1994 | 40 | 1069 | — | 414 | 322 |
| #2 | | | | | | | | | | |

In addition, all 24 treatments, including the control, received the antibiotic Lactrol (provided by Phibro) to prevent bacterial growth and competition. All enzymes were volumetrically diluted 2-fold based on weight to ensure precise delivery to each Labomat beaker. Once all components were added to the beakers, the sealed beakers were placed into the Labomat and were incubated for about 24 hours at 50° C., after which the samples were cooled to 40° C. The Labomat was programmed to rotate at 50 RPM and reverse direction of the wheel supporting the Labomat beakers every 50 seconds to improve mixing efficiency.

Once the Labomat beakers cooled from the saccharification step, the contents of each Labomat beaker (i.e., the saccharified liquefact) were transferred to a sterile 250 mL Erlenmeyer flask. Each flask was weighed prior to the addition of the liquefact and weighed after the addition of the liquefact to ascertain the exact mass that was contained within each flask. In addition, all replicates received 375 microliters (4) of sulfuric acid to adjust the pH of each sample flask to between 4.5-5.20. Urea was also added as a sterile 0.2 g/mL solution to a final concentration of 500 ppm (w/w) based on the nitrogen content of the urea (w/w, based on the total mass of liquefact).

In addition, further enzymes were added to the flasks to provide for additional saccharification during the fermentation process. Specifically, a conventional glucoamylase enzyme (Spirizyme Excel, provided by Novozymes) was prepared as a 0.25 g/mL solution and added at a dose of 0.066% (w/w), based on the wet weight of corn) to the eight control replicates. The remaining 16 experimental replicates received a different glucoamylase enzyme (provided by Novozymes, catalog number 22035) prepared as a 0.50 g/mL solution at a dose of 0.4% (w/w). This solution was incubated and mixed for 30 minutes at 40° C. before inoculation into a plurality of fermentation flasks. Each fermentation flask was inoculated with 322 μL of a yeast suspension (Ethanol Red; provided by Fermentis) to attain an initial concentration of 1×10⁷ yeast cells/mL. The mass of each flask was recorded after all additions, then sanitized fermentation traps were inserted into each flask to allow for the release of gaseous products in a sanitary manner. The flasks were incubated at 32° C. with shaking at 160 RPM in an incubator/shaker for a 107 hours.

Time point samples were taken at 46 hours and 107 hours and analyzed for the presence of ethanol as well the concentrations of substrates (glucose, DP2, DP3, DP4+, and xylose, where “DPx” represent glucose oligomers with “x” subunits) and products (ethanol, glycerol, lactic acid, and acetic acid) by HPLC. Fermentation progress was monitored by weighing the fermentation flasks periodically during the 4.5 day (i.e., 107 hours) incubation period. Samples were prepared for HPLC by centrifugation to remove large solids, followed by filtration through 0.45 μm syringe filters, and acidification to a pH of approximately 2 by the addition of sulfuric acid to a final concentration of 0.01 N. The final concentrations of total dry solids and dissolved dry solids, and the density of the filtrate were measured after incubation for each replicate. The initial amount of starch in the ground corn and the amount in the bottom of the flasks after fermentation was measured using the Megazyme Total Starch Assay Kit (Megazyme International). The bottoms of the flasks were prepared for analysis by combining the replicates of each treatment.

Results

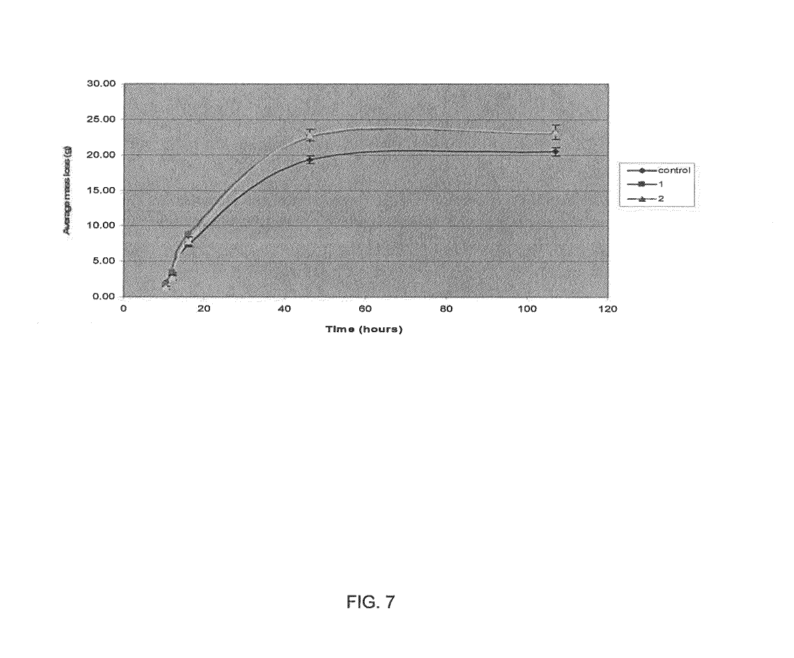

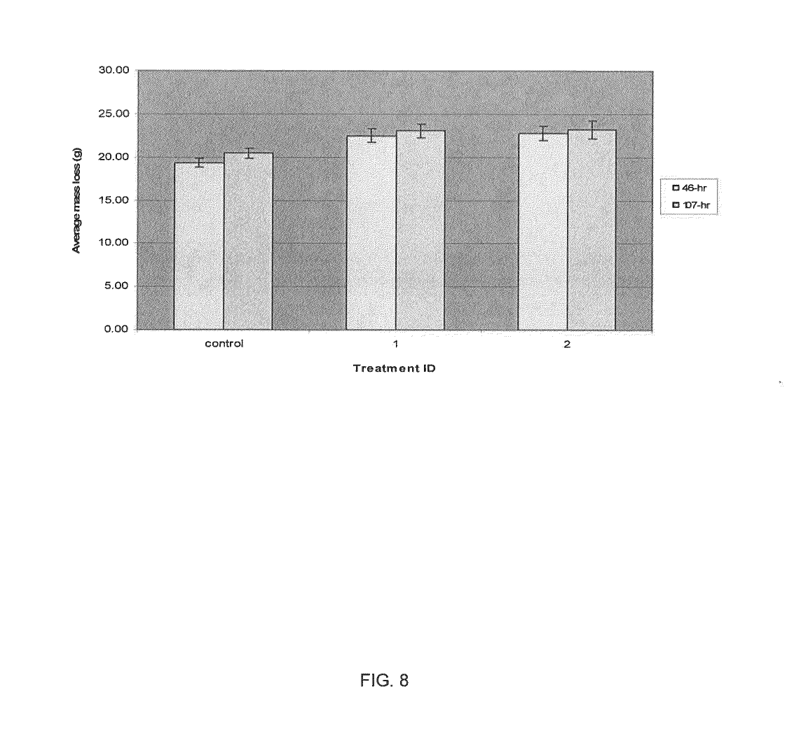

Initially, the general extent of the reaction was assessed via a determination of mass loss. Referring to FIG. 7, the reaction extent was estimated by using the mass loss measured at different time points. Specifically, fermentation progress was assessed by measuring the average mass loss in grams during the reaction (i.e., by weighing the reaction vessels or flasks). There was a significant difference in the average mass loss at the 16 hr time point among all treatments using analysis of variance (ANOVA) with a p value of less than 0.05 (p=5.95×10⁻⁶). In addition, there were also significant differences observed in the average mass loss at the 46 hr time point (p=3.31×10−9) and at the end of fermentation (107 hr) among all treatments, using ANOVA (p=7.45×10−7). The average mass loss at 46 hours and 107 hours is shown in FIG. 8 and Table 10. Accordingly, the data supports the conclusion that liquefaction, saccharification, and fermentation steps resulted in mass loss during the reaction. Moreover, the data in FIG. 8 illustrates that greater mass loss occurred in the two experimental treatments, relative to the control group.

TABLE 10
Summary of fermentation progression expressed as average mass loss
(expressed in grams)

| | Fermentation time (hrs) | | | | |
|---|---|---|---|---|---|
| Treatment ID | 10.5 | 12 | 16 | 46 | 107 |
| control | 2 ± 0.09 | 3.28 ± 0.14 | 7.34 ± 0.3 | 19.33 ± 0.54 | 20.47 ± 0.56 |
| 1 | 1.83 ± 0.11 | 3.47 ± 0.19 | 8.85 ± 0.42 | 22.52 ± 0.76 | 23.05 ± 0.76 |
| 2 | 1.33 ± 0.21 | 2.68 ± 0.31 | 7.77 ± 0.62 | 22.79 ± 0.83 | 23.2 ± 1.01 |

Next, the reactions also successfully produced ethanol. First, Table 11 illustrates the concentration and yields of ethanol within the flasks during the course of the fermentation reaction for all thee of the groups (i.e., one control and two treatment groups). Statistical analysis using ANOVA reveals significant differences in ethanol concentration between all of the treatment groups after both 46 hours (p=1.45×10⁻¹¹) and 107 hours (p=1.36×10⁻¹¹) of fermentation. Similarly, significant differences were also seen in the ethanol yield after both 46 hours (p=1.94×10⁻⁸) and 107 hours (p=1.41×10⁻⁵) of fermentation.

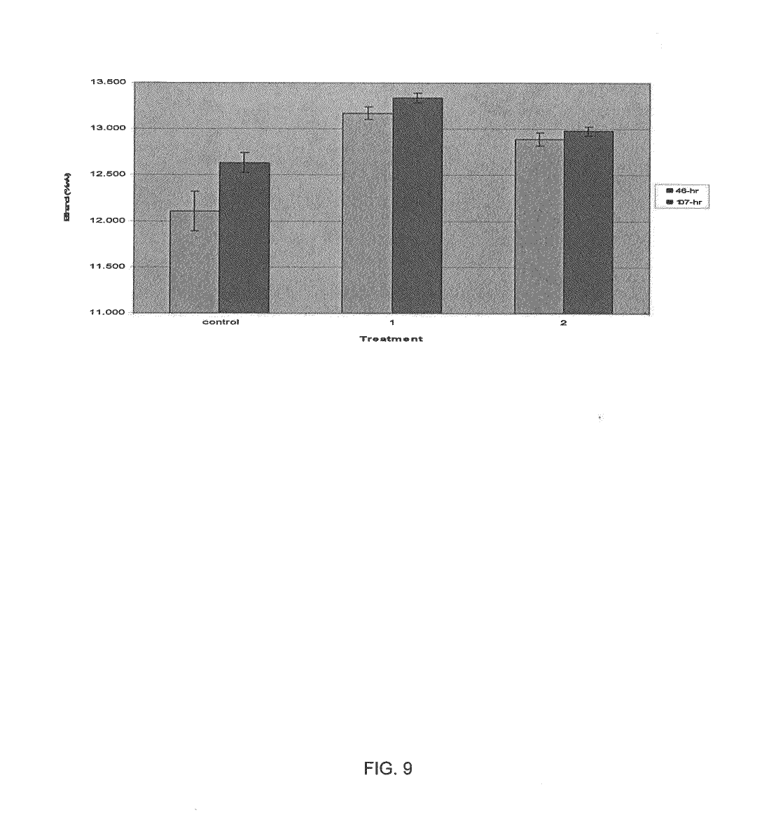

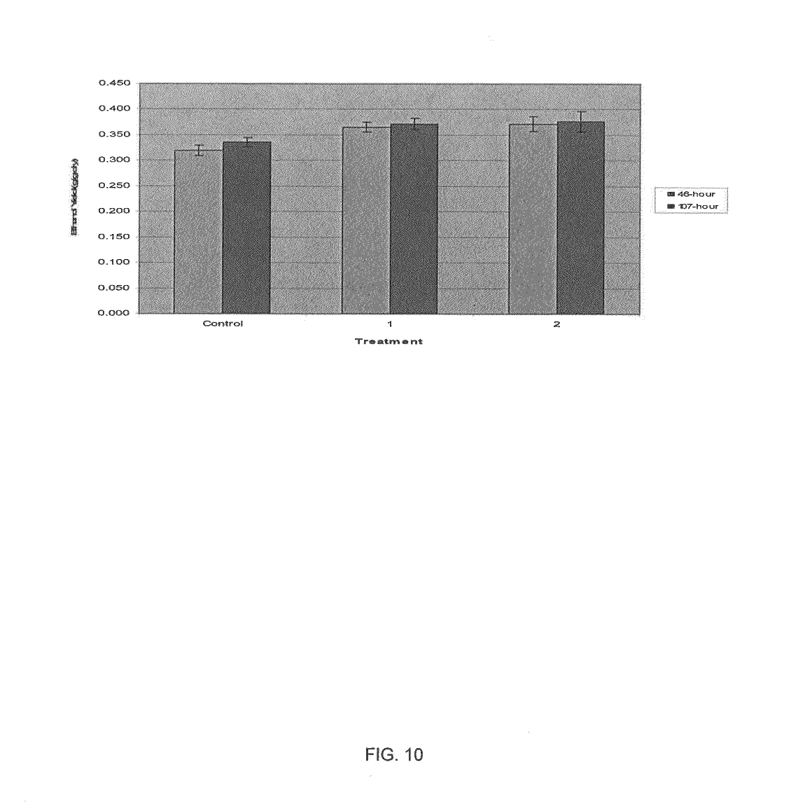

In addition, the same data is illustrated in FIGS. 9 and 10. Specifically, significant increases were seen in the ethanol concentrations at both 46 hours and 107 hours in the two treatment groups relative to the control group, as shown in FIG. 9. In particular, it appears that after both 46 and 107 hours of fermentation, the first treatment group produced a greater concentration of ethanol relative to the second treatment group. In addition, similar increases in ethanol yield were also seen in the treatment groups relative to the control group, as illustrated in FIG. 10. Accordingly, the above discussed data support the idea that both treatment regimens tested in these experiments lead to increased ethanol production relative to conventional production methods (i.e., the set-up used in the control group).

TABLE 11
Summary of ethanol concentration and ethanol yield at the 46-hr and 107-hr
time points.

| | 46-hr EtOH | 107-hr EtOH | 46-hr EtOH | 107-hr EtOH |
|---|---|---|---|---|
| Treatment ID | Concentration (% w/v) | Concentration (% w/v) | Yield (g/g dry corn) | Yield (g/g dry corn) |
| Control | 12.11 ± 0.22 | 12.64 ± 0.104 | 0.32 ± 0.01 | 0.336 ± 0.009 |
| 1 | 13.17 ± 0.07 | 13.34 ± 0.052 | 0.366 ± 0.01 | 0.372 ± 0.011 |
| 2 | 12.89 ± 0.07 | 12.98 ± 0.05 | 0.372 ± 0.015 | 0.379 ± 0.015 |

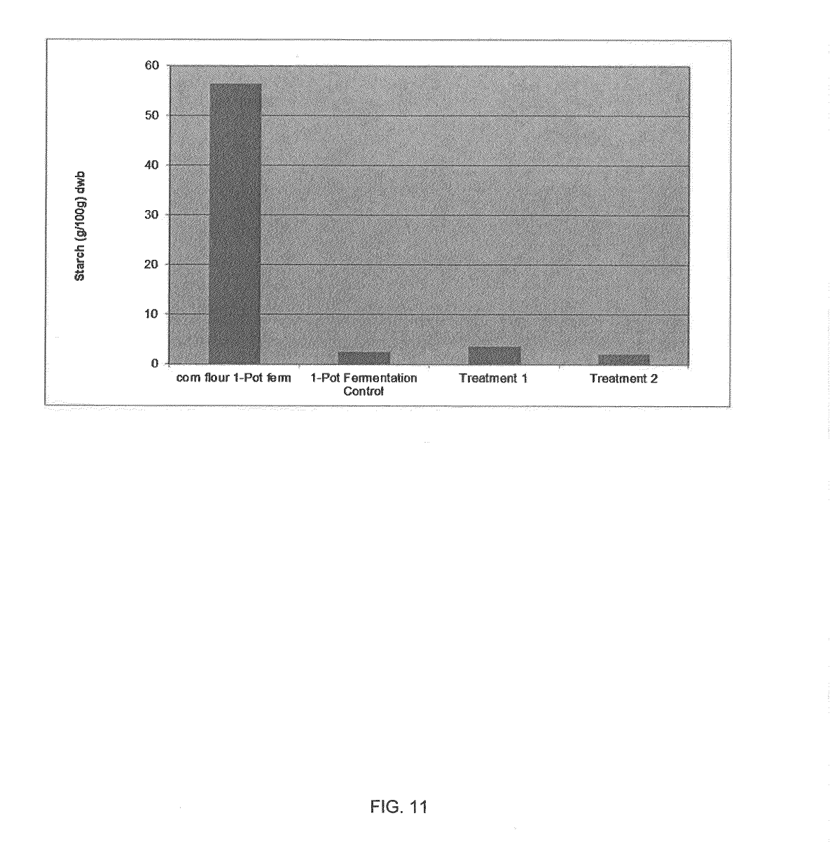

Next, starch consumption on a dry-weight basis (dwb) was assessed to see which groups consumed or broke down the greatest amount of starch during fermentation. As shown in FIG. 11, each of the groups (excluding the negative control group labeled “corn flour—one pot ferm,” which did not undergo fermentation) consumed a significant amount of starch during the fermentation reaction. Specifically, the second treatment group appears to be more effective at consuming starch than the control group.

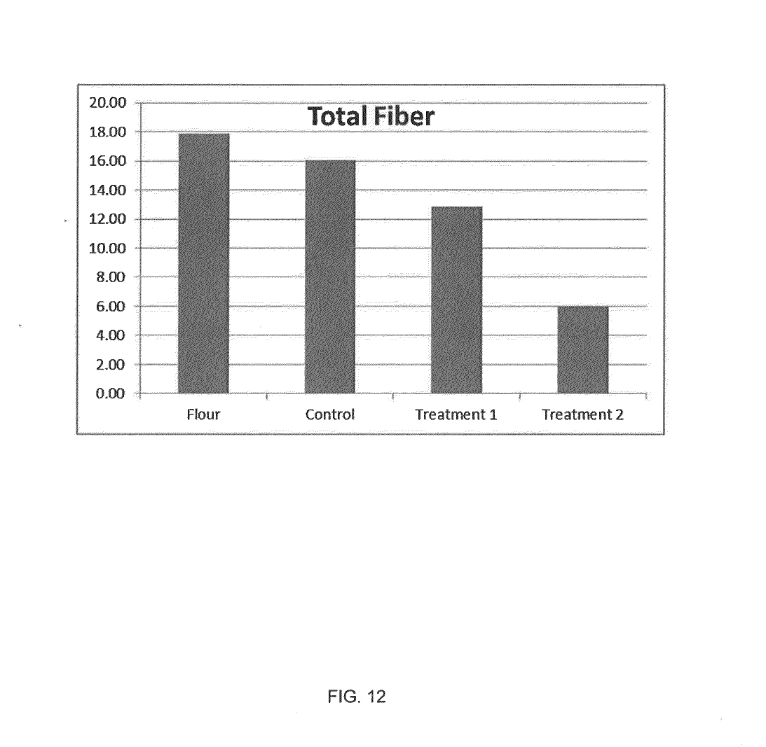

Similarly, the concentration of the total fiber content was also assessed to determine how well the treatments broke down the ground corn or corn flour during the experimental reactions. In particular, the concentrations of acid detergent fiber (ADF) and neutral detergent fiber (NDF) were assessed, as shown in FIG. 12. These data show that both treatments one and two were more effective at breaking down the ground corn than the control, with the second treatment being better at reducing the total fiber concentration than the first treatment group.

Accordingly, the above data show that different treatments can be used to successfully produce ethanol from a ground-corn starting material. In particular, the second treatment group showed improved amounts of starch and fiber breakdown relative to the first treatment and the control group, while the first group showed greater concentrations of ethanol production during the fermentation reaction.

It should be understood from the foregoing that, while particular embodiments have been illustrated and described, various modifications can be made thereto without departing from the spirit and scope of the invention as will be apparent to those skilled in the art. Such changes and modifications are within the scope and teachings of this invention as defined in the claims appended hereto.

What is claimed is:

1. A process for the production of ethanol, the processing comprising:
performing a liquefaction step, the liquefaction step comprising:
disposing an untreated carbon-containing material within a first vessel,
adding at least one enzyme to the first vessel, and
incubating the first vessel at a first temperature;
performing a saccharification step, the saccharification step comprising:
adding at least one enzyme to the first vessel, and
incubating the first vessel at a second temperature to form a liquefact within the first vessel; and
performing a fermentation step, the fermentation step comprising:
removing the liquefact from the first vessel,
disposing the liquefact in a second vessel,
adding at least one enzyme,
adding a plurality of yeast cells to the second vessel, and
incubating the second vessel at a third temperature.

2. The process of claim 1 further comprising adding a volume of a liquid to the first vessel during the liquefaction step.

3. The process of claim 1 further comprising adding at least one nutrient to the second vessel during the fermentation step.

4. The process of claim 1, wherein the untreated carbon-containing material is selected from the group consisting of a corn-based product, grass, wood, woody biomass, forage grasses, herbaceous energy crops, non-woody plant biomass, agricultural wastes, agricultural residues, forestry residues, forestry wastes, paper-production sludge, waste paper sludge, waste-water-treatment sludge, municipal solid waste, and sugar-processing residues.

5. The process of claim 1, wherein the at least one enzyme added in the liquefaction step is an alpha-amylase enzyme.

6. The process of claim 1, wherein the first temperature and the second temperature are between about 50° to about 100° Celsius.

7. The process of claim 1, wherein the first temperature and the second temperature are equal.

8. The process of claim 1, wherein the liquefaction and saccharification steps occur at substantially the same time.

9. The process of claim 1, wherein the at least one enzyme added during the saccharification step is selected from the group consisting of a cellulase complex, a β-glucanase, a xylanase, a hemicellulose, and combinations thereof.

10. The process of claim 9, wherein the at least one enzyme added during the saccharification step comprise a cellulase complex and a β-glucanase.

11. The process of claim 1, wherein the liquefaction and saccharification steps further comprise stirring the slurry with a mechanical actuating apparatus to form the liquefact.

12. The process of claim 1, wherein the liquefaction and saccharification steps are performed for a pre-determined period of time.

13. The process of claim 12, wherein the pre-determined period of time is about 24 hours.

14. The process of claim 1, wherein the third temperature is lower than the first temperature and the second temperature.

15. The process of claim 1, wherein the at least one nutrient comprises at least one of thiamine, urea, nicotinic acid, and ammonium chloride.

16. The process of claim 1, wherein the yeast cells are selected from the group consisting of an industrial grade yeast strain, a genetically-modified yeast strain, a Saccharomyces cerevisiae lineage yeast, and combinations thereof.

17. The process of claim 1, wherein the yeast cells are capable of fermenting xylose.

18. The process of claim 15, wherein the yeast cells are capable of fermenting glucose.

19. A process for the production of ethanol, the processing comprising:
performing a liquefaction step, the liquefaction step comprising:
disposing an untreated carbon-containing material within a first vessel,
adding a volume of a liquid to the first vessel;
adding at least one enzyme to the first vessel, and
incubating the first vessel at a first temperature to form a slurry;
performing a saccharification step simultaneous to the liquefaction step, the saccharification step comprising:
adding at least one enzyme to the first vessel,
stirring the slurry, and
incubating the first vessel at a second temperature to form a liquefact within the first vessel; and
performing a fermentation step, the fermentation step comprising:
removing the liquefact from the first vessel,
disposing the liquefact in a second vessel,
adding at least one enzyme to the second vessel,
adding at least one nutrient to the second vessel,
adding a plurality of yeast cells to the second vessel, and
incubating the second vessel at a third temperature.

20. A system for the production of ethanol, the system comprising:
a first vessel comprising a mechanical actuating apparatus operated at a first temperature and a second temperature for performing liquefaction and saccharification; and
a second vessel connected to the first vessel and operated at a third temperature for performing fermentation,
wherein an untreated carbon-containing material is disposed in the first vessel with a plurality of enzymes and incubated at the first and second temperatures to form a liquefact; and
wherein the liquefact is disposed in the second vessel with at least one enzyme, at least one nutrient, and a plurality of yeast cells and incubated at the third temperature.
